(19) United States
(12) Patent Application Publication
(10) Pub. No.: US 2026/0007451 A1
(43) Pub. Date: Jan. 8, 2026

(54) ELECTROSURGICAL SYSTEM WITH FREQUENCY-BASED CONTROL

(71) Applicant: Boston Scientific Scimed, Inc., Maple Grove, MN (US)

(72) Inventors: Steven Kinio, Pickering (CA); Matthew Protas, Blaine, MN (US); Cynthia Leung, Toronto (CA); Laurentiu Murtescu, Vaughn (CA); Deep Patel, Waterdown (CA)

(21) Appl. No.: 19/259,892

(22) Filed: Jul. 3, 2025

Related U.S. Application Data
(60) Provisional application No. 63/668,100, filed on Jul. 5, 2024.

(51) Int. Cl. A61B 18/12; A61B 18/00; A61B 18/14

(52) U.S. Cl. CPC A61B 18/1206; A61B 18/1492; A61B 2018/00642; A61B 2018/00684; A61B 2018/00702; A61B 2018/00732; A61B 2018/0075; A61B 2018/144

(57) ABSTRACT

An electrosurgical generator and system include device terminals including an active terminal and a return terminal and a controller. The controller adjusts an amplitude of a radiofrequency (RF) energy signal provided to the active terminal. The adjusted amplitude is based on a setpoint and an input in a closed feedback loop. The input is based on a rolling sum of addends, and each addend is based on a sampled output of the RF energy signal associated with a frequency bin. The rolling sum of addends is determined over a number of frequency bins associated with a target frequency.

This application claims priority to U.S. Provisional Patent Application No. 63/668,100 entitled “ELECTROSURGICAL SYSTEM WITH FREQUENCY-BASED CONTROL,” filed Jul. 5, 2024, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical units, such as radiofrequency (RF) generators, electrosurgical systems, and methods, that provide RF energy signals for electrosurgery including for tissue punctures, such as transseptal punctures in cardiac procedures.

### BACKGROUND

Catheters are often used to provide general access into a patient's body using minimally invasive techniques. In some examples, a catheter can be used to create a channel through a region of the body. For instance, punctures in tissues can provide access for medical tools used in various medical interventions. In one example, a pericardium layer of a patient can be punctured to provide for epicardial access, such as to create an access point to insert tools for epicardial ablation. In another example, electrosurgical devices are applied to remove accumulation of atheromatous material on the inner walls of vascular lumens, which results in atherosclerosis. In one technique, an electrosurgical device is applied to puncture through the vascular occlusion without affecting the vessel walls. Another example is a transseptal puncture in a cardiac procedure. The left atrium is a difficult cardiac chamber to reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium.

Punctures, such as transseptal punctures, can be performed with the aid of guidewires having electrodes energized with a suitable power source such as an electrically coupled power generator to provide the source of RF energy in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver the RF energy to the area where tissue is to be affected. Electrosurgical devices pass RF energy through tissue between the electrodes to puncture tissue with plasma formed on the energized electrode. Tissue that contacts the plasma experiences a rapid vaporization of cellular fluid to produce a cutting effect. Electrical energy can be applied to the electrodes either as a train of high frequency pulses or as a continuous signal typically in the radiofrequency (RF) range to perform the cutting or puncturing techniques.

### SUMMARY

In Example 1, an electrosurgical generator, comprising: a plurality of device terminals including an active terminal and a return terminal; and a controller configured to: adjust an amplitude of a radiofrequency (RF) energy signal provided to the active terminal, the adjusted amplitude based on a setpoint and an input in a closed feedback loop, wherein the input is based on a rolling sum of a plurality of addends, each addend of the plurality of addends based on a sampled output of the RF energy signal associated with a frequency bin, the rolling sum of addends determined over a number of frequency bins associated with a target frequency.

In Example 2, the electrosurgical generator of Example 1, wherein the controller further includes a feedback control device to receive an error based on the setpoint and the input.

In Example 3, the electrosurgical generator of Example 2, wherein the feedback control device includes a proportional-integral-derivative (PID) mechanism.

In Example 4, the electrosurgical generator of any of Examples 2 and 3, wherein the feedback control device is configured to adjust the amplitude of the RF energy signal.

In Example 5, the electrosurgical generator of any of Examples 1-4, wherein the frequency bin and the number of frequency bins are applied to determine the target frequency.

In Example 6 the electrosurgical generator of Example 5, wherein the frequency bin and the number of frequency bins are applied to determine a sampling rate of the sampled output.

In Example 7, the electrosurgical generator of any of Examples 1-6, wherein the frequency bin and the number of frequency bins are applied to determine sinusoidal data applied to the sampled output to determine each addend.

In Example 8, the electrosurgical generator of Example 7, wherein the sinusoidal data is received from a look up table.

In Example 9, the electrosurgical generator of Example 8, wherein the look up table includes a cycle of sinusoidal data.

In Example 10, the electrosurgical generator of any of Examples 1-9, wherein the rolling sum of the plurality of addends is based on the plurality of addends determined over time.

In Example 11, the electrosurgical generator of Examples 10, wherein the plurality of addends is equal to the number of frequency bins.

In Example 12, the electrosurgical generator of any of Examples 1-11, wherein the plurality of addends and the rolling sum of plurality of addends are received from a rolling accumulation buffer.

In Example 13, the electrosurgical generator of any of Examples 1-12, wherein the input includes a magnitude and phase based on the rolling sum of the plurality of addends.

In Example 14, the electrosurgical generator of Examples 13, wherein the magnitude is a root-mean-square of the rolling sum of the plurality of addends.

In Example 15, the electrosurgical generator of any of Examples 13 and 14, wherein the phase is an arctangent of the rolling sum of the plurality of addends.

In Example 16, an electrosurgical generator, comprising: a plurality of device terminals including an active terminal and a return terminal; and a controller configured to: adjust an amplitude of a radiofrequency (RF) energy signal provided to the active terminal, the adjusted amplitude based on a setpoint and an input in a closed feedback loop, wherein the input is based on a rolling sum of a plurality of addends, each addend of the plurality of addends based on a sampled output of the RF energy signal associated with a frequency bin, the rolling sum of addends determined over a number of frequency bins associated with a target frequency.

In Example 17, the electrosurgical generator of Example 16, wherein the controller further includes a feedback control device to receive an error based on the setpoint and the input.

In Example 18, the electrosurgical generator of Example 17, wherein the feedback control device includes a proportional-integral-derivative (PID) mechanism.

In Example 19, the electrosurgical generator of Example 17, wherein the feedback control device is configured to adjust the amplitude of the RF energy signal.

In Example 20, the electrosurgical generator of Example 16, wherein the frequency bin and the number of frequency bins are applied to determine the target frequency.

In Example 21, the electrosurgical generator of Example 20, wherein the frequency bin and the number of frequency bins are applied to determine a sampling rate of the sampled output.

In Example 22, the electrosurgical generator of Example 16, wherein the frequency bin and the number of frequency bins are applied to determine sinusoidal data applied to the sampled output to determine each addend.

In Example 23, the electrosurgical generator of Example 22, wherein the sinusoidal data is received from a look up table.

In Example 24, the electrosurgical generator of Example 23, wherein the look up table includes a cycle of sinusoidal data.

In Example 25, the electrosurgical generator of Example 16, wherein the rolling sum of the plurality of addends is based on the plurality of addends determined over time.

In Example 26, the electrosurgical generator of Example 25, wherein the plurality of addends is equal to the number of frequency bins.

In Example 27, the electrosurgical generator of Example 16, wherein the plurality of addends and the rolling sum of the plurality of addends are received from a rolling accumulation buffer.

In Example 28, the electrosurgical generator of Example 16, wherein the input includes a magnitude and phase based on the rolling sum of the plurality of addends.

In Example 29, the electrosurgical generator of Example 28, wherein the magnitude is a root-mean-square of the rolling sum of the plurality of addends.

In Example 30, the electrosurgical generator of Example 29, wherein the phase is an arctangent of the rolling sum of the plurality of addends.

In Example 31, an electrosurgical system, comprising: an electrosurgical device having an active electrode; and an electrosurgical generator, comprising: a plurality of device terminals including an active terminal and a return terminal, the active electrode configured to electrically couple to the active electrode; and a controller configured to: adjust an amplitude of a radiofrequency (RF) energy signal provided to the active terminal, the adjusted amplitude based on a setpoint and an input in a closed feedback loop, wherein the input is based on a rolling sum of a plurality of addends, each addend of the plurality of addends based on a sampled output of the RF energy signal associated with a frequency bin, the rolling sum of addends determined over a number of frequency bins associated with a target frequency.

In Example 32, the electrosurgical system of Example 31, wherein the electrosurgical device includes a crossing device.

In Example 33, the electrosurgical system of Example 32, wherein the crossing device includes a transseptal guidewire.

In Example 34, a method for use with an electrosurgical generator having a plurality of device terminals including an active terminal, the method comprising: adjusting an amplitude of a radiofrequency (RF) energy signal provided to the active terminal, the adjusted amplitude based on a setpoint and an input in a closed feedback loop; and wherein the input is based on a rolling sum of a plurality of addends, each addend of the plurality of addends based on a sampled output of the RF energy signal associated with a frequency bin, the rolling sum of addends determined over a number of frequency bins associated with a target frequency.

In Example 35, the method of Example 34, and further comprising receiving sinusoidal data from a lookup table, the sinusoidal data applied to the sample output to determine each addend.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a block diagram illustrating an example electrosurgical generator configured for use with the electrosurgical system of FIG. 1.

FIG. 3 is a block diagram illustrating an example closed feedback control system of the example electrosurgical generator of FIG. 2.

FIG. 4 is a block diagram illustrating a process to configure the closed feedback control system of FIG. 3.

FIG. 5 is a block diagram illustrating a process of the closed feedback control system of FIG. 3 configured according to the process of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

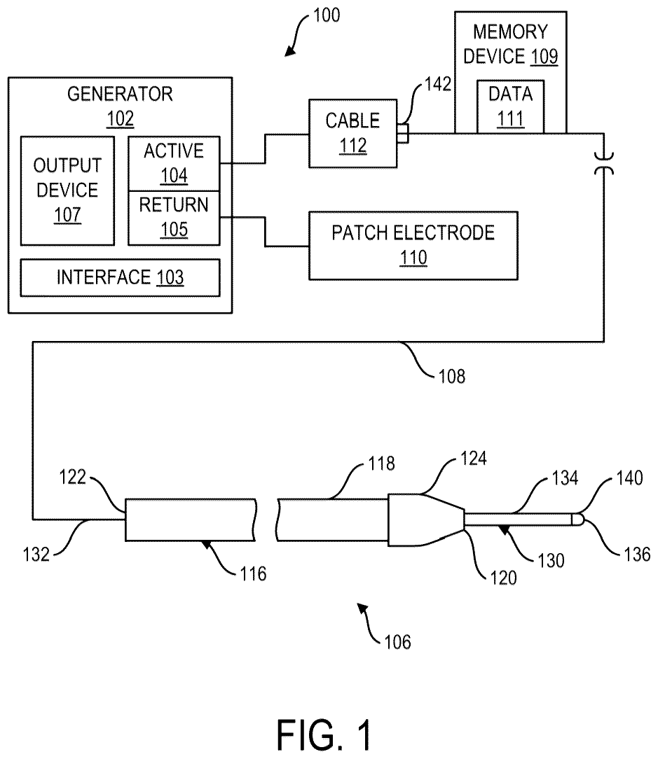

FIG. 1 illustrates an embodiment of an electrosurgical system 100 to facilitate vascular access to a heart and provide catheter positioning within cardiac anatomy. The embodiment of the medical system 100 includes a radiofrequency (RF) electrosurgical unit, such as an RF electrosurgical generator 102 and an electrosurgical device, such as an electrosurgical crossing device 106 to puncture tissue in the illustrated example. In one example, the electrosurgical crossing device 106 includes an electrosurgical transseptal guidewire 108. In the illustration, the electrosurgical transseptal guidewire 108 is electrically coupled to the electrosurgical generator 102 via cable 112, such as to an active connector 104 on the electrosurgical generator 102. The electrosurgical generator 102 is configured to provide a source of energy, such as RF energy to the electrosurgical transseptal guidewire 108 via the cable 112. In some embodiments, the system 100 includes a ground pad electrode, or indifferent (dispersive) patch electrode 110 electrically coupled to the electrosurgical generator 102, such as to a return connector 105 on the electrosurgical generator 102, for use with the electrosurgical transseptal guidewire 108 in a monopolar configuration. In some embodiments, the electrosurgical transseptal guidewire is implemented in a bipolar configuration using a pair of electrodes on the guidewire and without a separate patch electrode.

The electrosurgical generator 102 is configured to provide the source of RF energy to the electrosurgical transseptal guidewire 108 for a puncture operation with the electrosurgical device 106. The electrosurgical generator 102 includes an interface 103 including a set of user accessible controls, device terminals such as active terminal 104 and a return terminal 105, and an output device 107 such as a display device, speakers, and lights. During a monopolar puncture operation of electrosurgical generator 102, a first electrode, often referred to as the active electrode, is electrically coupled to the active terminal and a second electrode, or return electrode, is electrically coupled to the return terminal of the electrosurgical generator 102. The active electrode is typically provided with the electrosurgical device 106 in general and with the transseptal guidewire 108 in the illustration while the second electrode, such as patch electrode 110, is typically located on the back, buttocks, upper leg, or other suitable anatomical location of the patient during surgery. In such a configuration, the patch electrode 110 is often referred to as a patient return electrode.

An electrical circuit of RF energy via an RF energy signal is formed between the active terminal 104 and the return terminal 105, or active electrode and the patch electrode 110 through the patient, which is used to puncture tissue at the active electrode. For example, RF energy for a puncture function in a monopolar mode may be provided via the RF energy signal at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 108. In one example, the electrosurgical generator 102 provides RF energy via the RF energy signal to the active electrode as an alternating current having a frequency in the range of 100 kHz to 10 MHz. Typically, this energy is applied in the form of a continuous sinusoidal energy signal. In some embodiments, the energy is applied in bursts of pulses. The individual pulses in each burst of a pulsed energy signal typically each have a duration of 300 milliseconds with an interval between pulses of 700 milliseconds but can vary such as based on parameters of the connected electrosurgical device 106. The actual pulses are often sinusoidal or square waves and bi-phasic, that is alternating positive and negative amplitudes.

In one example, the electrosurgical generator 102 provides the power to the electrosurgical puncture device 106, but the actual power level delivered to the electrosurgical puncture device 106 can be selected via controls on the electrosurgical puncture device 106 rather than controls on the electrosurgical generator 102. In another example, the electrosurgical generator 102 can be programmed to provide power levels within a selected range of power, and the electrosurgical puncture device 106 is used to select an output power level within the preprogrammed range. For instance, the electrosurgical generator 102 can be programmed to provide monopolar energy for a puncture function in a first range of power settings as well as voltage-based controls to target a specific voltage. The electrosurgical generator 102 can be programmed to provide monopolar energy for another function in a second range of power or voltage settings, which second range may be the same as, different than, or overlap the first range. In some embodiments, the user may then select the function and adjust the power or voltage setting within the range using controls on the electrosurgical puncture device 106 rather than using controls on the electrosurgical generator 102.

In one embodiment, the electrosurgical generator 102 can program and select particular controls, or ranges of controls, based on the particular configuration of the electrosurgical transeptal guidewire 108. The transseptal guidewire of the embodiment includes a memory device 109 (non-transitory memory) storing a set of parameters 111 associated with the transseptal guidewire 108. The electrosurgical generator 102 is configured to read the parameters to program the controls to be suited for the associated transseptal guidewire. The memory device 109 can store the parameters 111 in various memory segments having lookup tables or other data structures to provide data to be loaded into a memory device in the electrosurgical generator 102 and read by a controller of the electrosurgical generator to affect operation. Example parameters 111 can include model number of the transseptal guidewire 108, acceptable power levels signals applied to the transseptal device 108, whether the transseptal device is configured for single use or multiple uses, as well as other parameters.

The illustrated electrosurgical puncture device 106 includes the electrosurgical transseptal guidewire 108 and a delivery component 116. While embodiments of the disclosure are described with reference to punctures in tissue with a transseptal guidewire for illustration, the features of the disclosure, such as the electrosurgical generator 102, can be used with other electrosurgical devices including other transseptal surgical devices such as needle-based platforms. The delivery component 116 includes an elongated shaft 118 having a shaft distal tip 120. The elongated shaft 118 defines a longitudinally extending axial lumen 122. The electrosurgical transseptal guidewire 108 is adapted to be disposed within the lumen 122 and coupled to the RF energy source. In some embodiments, the delivery component 116 can include an elongate sheath, and the electrosurgical transseptal guidewire 108 is disposed within the sheath. In another embodiment, the delivery component 116 can include a dilator/sheath assembly, and the electrosurgical transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE). The shaft 118 and distal tip can include various materials such as metal hypotubes as well.

The electrosurgical transseptal guidewire 108 includes a puncture wire shaft 130 with a puncture wire proximal portion 132 and a puncture wire distal portion 134 having a puncture wire distal tip 136. The puncture wire distal tip 136 includes a puncture electrode 140 adapted to deliver the RF energy. The puncture electrode 140 is configured as the active electrode. The puncture wire proximal portion 132 includes an end connector 142 configured to electrically couple to cable 112 and receive an RF signal from the electrosurgical generator 102. In one example, the electrosurgical transeptal guidewire 108 can be coupled to and uncoupled from the cable 112 depending on whether the electrosurgical transeptal guidewire 108 is used as an electrosurgical puncture device or as an exchange rail, for instance. The transseptal guidewire 108 is configured to conduct the RF signal from the proximal portion 132 along the puncture wire shaft 130 to the electrode 140. In some embodiments, the puncture wire shaft 130 is constructed from an electrically conductive material having an insulative outer coating. In some embodiments, the electrically conductive material is a flexible, shape memory material such as a nickel titanium alloy or nitinol. The exposed electrode 140 is configured to apply the RF energy, such as to puncture tissue.

In the illustrated example, the electrosurgical transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 108 performs multiple functions and reduces the amount of device exchanges in the medical procedure. The transseptal guidewire 108 includes a distal tip 136 extendable from the delivery component distal end 120 such that the delivery component 116 is retractable from the patient over the guidewire 108 with the guidewire distal tip 136 disposed within the heart. The transseptal guidewire 108 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 116, the distal portion 134 is biased to form a coil for anchoring the transseptal guidewire 108 beyond the puncture. The delivery component 116 is retractable from the patient over the transseptal guidewire 108 with the distal tip 136 still disposed within the heart. The transseptal guidewire 108 can also support the installation of therapy devices to a therapy location in the patient's heart, such as tubular members or other catheters and for advancing other devices within the heart.

In an anticipated use of the system 100, the electrosurgical device 106 is coupled to the RF generator 102, and if the electrosurgical device 106 is to be configured in a monopolar mode, the patch electrode 110 is coupled to the patient. The RF generator 102 can be set to a puncture mode, such as an energy output of approximately 10 watts. In some examples, femoral access is obtained via a conventional percutaneous needle, and the transseptal guidewire 108 is inserted into the vasculature and advanced to the superior vena cava. The shaft distal tip 120 of the delivery component 116 is advanced over the proximal portion 132 of the guidewire 108, and the distal tapered portion 124 of the delivery component shaft 118 is advanced over the guidewire 108 to the superior vena cava. Under visualization, the distal tapered portion 124 is moved from the superior vena cava to the right atrial septum and then to the fossa ovalis of the heart. Once the delivery component distal tip 120 is confirmed at the fossa ovalis, the electrode 140 of the transseptal guidewire 108 is advanced from the delivery component distal tip 120. In one example, the exposed puncture electrode 140 of the transseptal guidewire 108 is extended a few millimeters from the delivery component distal tip 120 to tent the heart tissue, and the transseptal guidewire 108 can be locked in position with respect to the delivery component 116. Forward pressure is applied to the electrosurgical device 106 and the transseptal guidewire 108 is actuated to apply the RF energy to the electrode 140 and puncture the fossa ovalis. The RF energy punctures the fossa ovalis and creates an aperture in the fossa ovalis. The transseptal guidewire 108 is unlocked from the delivery component 116, and the transseptal guidewire 108 is extended through the aperture. In general, the transseptal guidewire 108 is extended longitudinally for several millimeters prior to the distal portion 134 curving to assume a J-tip or pigtail shape and deflecting away from the atrial septum. The transseptal guidewire 108 can be advanced into the left atrium of the heart and anchored. In the embodiment of the delivery component 116 configured as the dilator/sheath assembly, the distal tapered portion 124 of a dilator, the distal tapered portion 124 is advanced into the puncture site to expand the aperture. The delivery component 116 can be retracted from the patient over the transseptal guidewire 108, and transseptal guidewire 108 can provide support for the installation of tubular members or other catheters and for advancing other devices within the heart.

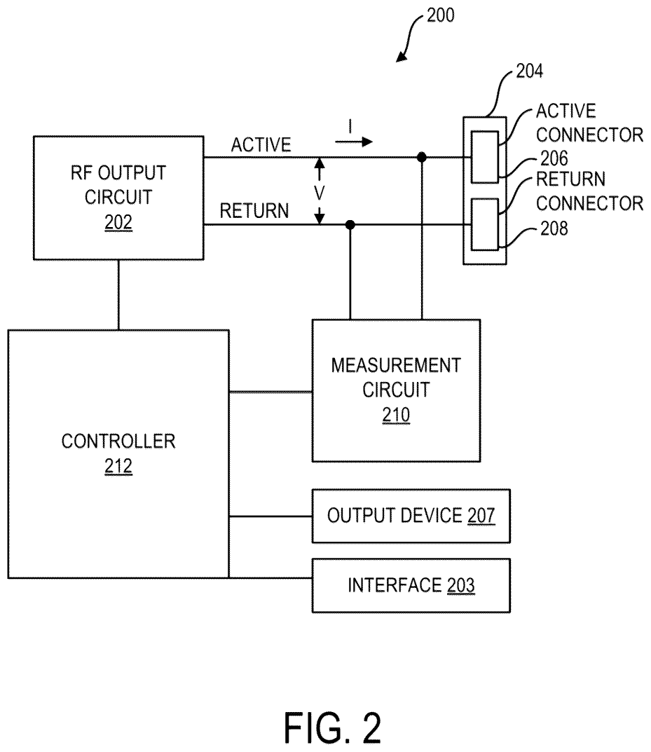

FIG. 2 illustrates an embodiment of an electrosurgical unit, such as an RF electrosurgical generator 200, which can correspond with electrosurgical generator 102 in system 100, to output an RF energy signal. The electrosurgical generator 200 includes an RF output circuit 202, a plurality of device terminals, such as device connectors 204 including an active terminal or active connector 206 and a return terminal or return connector 208, a measurement circuit 210, a controller 212, an output device 207 and an interface 203. The output device 207 and interface 203, in one embodiment, corresponds with output device 107 and interface 103, respectively.

In one embodiment, the RF output circuit 202 is configured to generate an output RF energy signal at a frequency. The RF output circuit 202 can include a power supply to provide a direct current signal and can convert the direct current signal to an alternating current signal. The RF output circuit 202 is configured to generate a plurality of voltages, waveforms having various duty cycles, peak voltages, crest factors, frequencies and other suitable parameters and provide, or output, the selected RF energy signal to the active connector 206. The device connectors 204 can be configured to include receptacles located on a housing of the RF generator 200 that can be mechanically coupled to electrosurgical devices. The device connectors 204 are configured to electrically couple the electrosurgical generator 200 to various electrosurgical devices. For example, the active connector 206 is suitable for electrically coupling to cable 112, which can be electrically coupled to the transeptal guidewire 108. The return connector 208 is suitable for electrically coupling to the ground pad electrode 110 when an electrosurgical device is operated in a monopolar mode or to a return electrode on the electrosurgical device when operated in a bipolar mode. The measurement circuit 210 is electrically coupled to the device connectors 204 and is configured to determine current or voltage measurements from RF energy signal generated by the RF output circuit 202 and present the current and voltage measurements to the controller 212. The measurement circuit 210 can include circuit elements or paths electrically coupled to the RF output circuit 202 or at least some of the output connectors 204 including the active connector 206 and return connector 408 and is configured to provide a signal representative of the active and return voltages and active current. In one embodiment, the measurement circuit 210 samples the RF energy signal at a selected frequency. The circuit elements can include current probes to measure currents of interest. In one embodiment, the measurement circuit 210 includes an analog to digital converter coupled to the circuit elements and the controller 212 to provide digital signals to the controller 212.

In embodiments, the controller 212 is implemented with any combination of hardware and programming to configure the functions of electrosurgical generator 200. In one embodiment, the programming for the electrosurgical generator 220 include processor executable instructions stored on at least one non-transitory machine-readable storage medium, such as a memory device and the hardware includes at least one processing resource, such as a microprocessor, to execute those instructions. In some embodiments, the hardware includes other electronic circuitry to at least partially implement at least one feature of electrosurgical generator 200. In some embodiments, the at least one machine-readable storage medium stores instructions that, when executed by the processor, at least partially implement some or all features of electrosurgical generator 200 and accesses data structures stored on a memory device coupled to the processor. In some embodiments, electrosurgical generator 200 includes the at least one machine-readable storage medium storing the instructions and the at least one processing resource to execute a method. The processor-executable instructions may be in the form of an application, such as a computer application or module of a computer application. The controller 212 in embodiments includes a processor operably connected to a memory device. The memory device can store processor executable instructions configured to control the processor, such as a program. Examples of a memory device can include a non-volatile memory device and a volatile memory device. Memory device can include various combinations of one or both of non-volatile memory devices and volatile memory devices.

In other embodiments, the functionalities of electrosurgical generator 200 and method may be at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, code, or pseudocode can be implemented in such electronic circuitry.

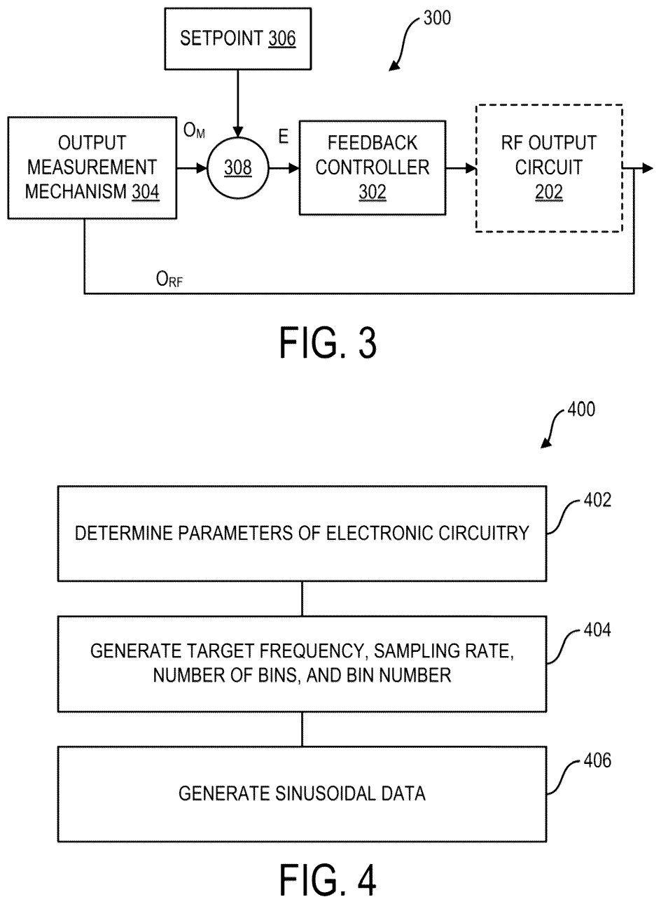

FIG. 3 illustrates an embodiment of a closed feedback loop controller 300 implemented in the electrosurgical generator 200 such as in controller 212. In a closed feedback loop, the output of a system, such as the RF energy signal output from the RF output circuit 202, is fed back through measurement device, such as measurement circuit 210, to digitize the output. The digitized output is processed, such as via controller 212, to generate a feedback input for a comparison with a references value to determine an error (difference between the feedback input and the reference value). A feedback controller 300 takes the error to change the system under control, such as the RF output circuit 202. The illustrated feedback loop controller 300 includes a feedback control device 302 and an output measurement mechanism 304. The output measurement mechanism 304 receives the output O_{RF} of a system, such as an amplitude (voltage) or electrical current information of the RF energy signal output from the RF output circuit 202, generates a mechanism output O_{M} (or feedback input), such as a sampled, processed, waveform amplitude and phase data, and compares the mechanism output O_{M} to a setpoint 306 via comparator 308 to generate an error E. The error E is provided to the feedback control device 302, to change the output O_{RF} under RF output circuit 202 control. In embodiments, the setpoint 306 is determined based on a user input or a configured input. In one example, the setpoint is selected from one or more available values based on one or more selectable RF energy signals. The feedback control device 302 and comparator 308 can be implemented with a control loop feedback mechanism such as a proportional-integral-derivative (PID) mechanism and constructed in the controller 212.

In example electrosurgical generators, the mechanism output O_{M} is typically based on a root-mean-square (RMS) or similar of the measured RF energy signal. Techniques for determining the RMS value range can include calculating the average sampled voltage of the output O_{RF} over a selected timespan. Often, this determination in electronic circuitry results in a loss of valuable contextual information stored in the energy signal waveform. Software solutions for averaging-based RMS calculations involve direct current (DC) offset issues and noise from harmonic signal.

In embodiments of the electrosurgical generator 200, the feedback loop controller 300 leverages a Fourier Transform process to generate a feedback control signal. For instance, the Fourier Transform allows an alternating current (AC) signal such as the RF energy signal to be decomposed into sinusoid frequency components, i.e.,

$$f ( t ) = {a}_{7} * sin ( {f}_{7} * t ) + {a}_{2} * sin ( {f}_{2} * t ) + {a}_{3} * sin ( {f}_{3} * t ) + {a}_{4} * sin ( {f}_{4} * t )$$

Data from the decomposed signals can provide information such as amplitude and phase from the signals. The feedback loop controller 300 incorporates a rolling, single frequency Discrete Fourier Transform (DFT)-based control implementation. For instance, the resulting Fourier Transform is used to convert measurements of the RF energy signal into frequency bins, such as a single frequency bin for the target RF frequency such as the frequency of the RF energy signal (O_{RF}). The frequency bin is applied to retrieve an RMS amplitude and phase information from voltage and current information from the measured RF energy signal. The DFT is updated for each sample. The embodiments of the feedback loop controller 300 include several advantages. Among the advantages, the use of the single frequency bin eliminates DC data and filters out noise from harmonics that leak into the measurement circuit 210 or feedback loop controller 300. Updating the DFT at each sample provides for fast data capture that can produce RMS information at the native sample rate of the hardware used to implement the feedback loop controller 300, such as field programmable gate array, which allows for high resolution of the RF energy signal. Further, the DFT produces real and imaginary components, which are converted into RMS magnitude and phase angle. Relative phase angle is determined via the difference between voltage and current phase angles.

FIG. 4 illustrates an example process 400 for configuration of the feedback loop controller 300, such as the implementation of the output measurement mechanism 304 to provide an input to the comparator 308. Design of the feedback loop controller 300 is based on such determined parameters as the target RF frequency range, or the desired frequency range of the RF energy signal, and the maximum delay time suitable for performing a process to update the DFT at 402. Based on parameters, a frequency bin number k and number of frequency bins N are determined at 404. The electronic circuitry to sample the RF energy signal is selected to generate a sample rate of at least twice the rate target RF frequency and the logic circuitry is capable of performing the process within a sample window. For example, if a 500-microsecond delay is a maximum acceptable value at a 1 MHz sampling rate, then the maximum number of frequency bins N is 500. The selected frequency bin is used to determine the frequency range of the RF energy signal. For example, values of bin number k and number of bins N, which are both integers, are selected to find matches for the following equation:

$$( ( k - 1 ) / N ) ⋆ {F}_{SAMPLE} = {F}_{RF}$$

where F_{SAMPLE} is the sampling rate and F_{RF} is the target, or selected, RF frequency. Accordingly, the sampling rate and the target RF frequency of the generator 200 can be based on the selected bin number k and number of bins N rather than vice versa. The selected bin number k and number of bins Nare used to generate sinusoidal data at 406.

In some embodiments, the feedback loop controller 300 includes, or has access to, sinusoidal look up tables (LUTs) based on the selected bin number k and number of bins N in a memory device to provide sinusoidal data at 406. Values in the sinusoidal look up tables are used in performing the process of the output measurement mechanism 304, and the use of LUTs can reduce time spent in operating the electronic circuitry of the output measurement mechanism 304 of the feedback loop controller 300. The LUTs map an input, such as an index value, to a corresponding output value to approximate a mathematical function. In a sine LUT, to be included in or accessed by the output measurement mechanism 304, each index value corresponds with an output value determined from

$$sin ( - 2 π * index * ( k - 1 ) / N )$$

In a cosine LUT, also to be included in or accessed by the output measurement mechanism 304, each index value corresponds with an output value determined from

$$cos ( - 2 π * index * ( k - 1 ) / N )$$

The output values are periodic. For instance, periodicity, or the completion of a period or cycle of output values, can be detected when at least two output values in a row match the first two output values. Once periodicity is detected, the overlapping output values can be eliminated from the table to create a cycle of output values corresponding with index values. In one embodiment, each sinusoidal LUT includes one cycle of output values. Periodicity occurs in at most 2*N index values, and in many cases, periodicity occurs in less than N index values. Because values of bin number k and number of bins N affect the sampling rate and target frequency and affect the output value determined from each sinusoidal LUT, each selected sampling rate or target frequency can involve a separate set of sinusoidal LUTs for the output measurement mechanism 304.

In some embodiments, the following pseudo code function describes an implementation a process to receive inputs of bin number k and number of bins N and generate a sinusoidal LUT at 406 in a data structure such as an array accessed by the output measurement mechanism 304.

```
function [out Array lookupTableOut] = buildDFTCosLookupTable (in k,
in N)
  counter = 1;
  %First two values are saved to determine periodicity
  cycleStartValue = cos(0);
  cycleSecondValue = cos(−2*pi*index*(k−1)/N);
  startFlag = false;
  currentValue = cycleSecondValue;
  lookupTableOut[0] = [cycleStartValue];
  while (index < N*2)
    %Check for periodicity and end of cycle
    if (startFlag && abs(currentValue − cycleSecondValue) < 1e−10)
    index = index − 1;
    exit loop;
    else
    startFlag = false;
    end if
    if (abs(currentValue − cycleStartValue) < 1e−10)
    startFlag = true;
    end if
    %Include an index/sinusoidal data pair into table
    lookupTableOut [index] = currentValue;
    index = index + 1;
    currentValue = cos(−2*pi*index*(k−1)/N);
  loop
end
```

The sinusoidal LUT generated with the example function is a cosine LUT in which each index value corresponds with an output value as determined above. A sine LUT can be generated by substituting cos( ) with sin( ) in the code. A cycle of values of generated, and periodicity is determined if two consecutive generated currentValue match the first two output values cyclestartValue and cycleSecondValue of the array. A match is determined if the two abs( ) approximates zero, in which abs( ) is the absolute value function. The example code approximate zero as a value less than 1⁽⁻¹⁰⁾, but other low values can be applied. Use of the approximate zero can be substituted for zero because of a rounding while using floating point logic. Each array includes at most N*2 index value/output value pairs, but a cycle of output values often includes less than N*2 output values and thus each array often includes less than N*2 index value/output value pairs. A LUT with a cycle of sinusoidal data includes values up to a detected periodicity.

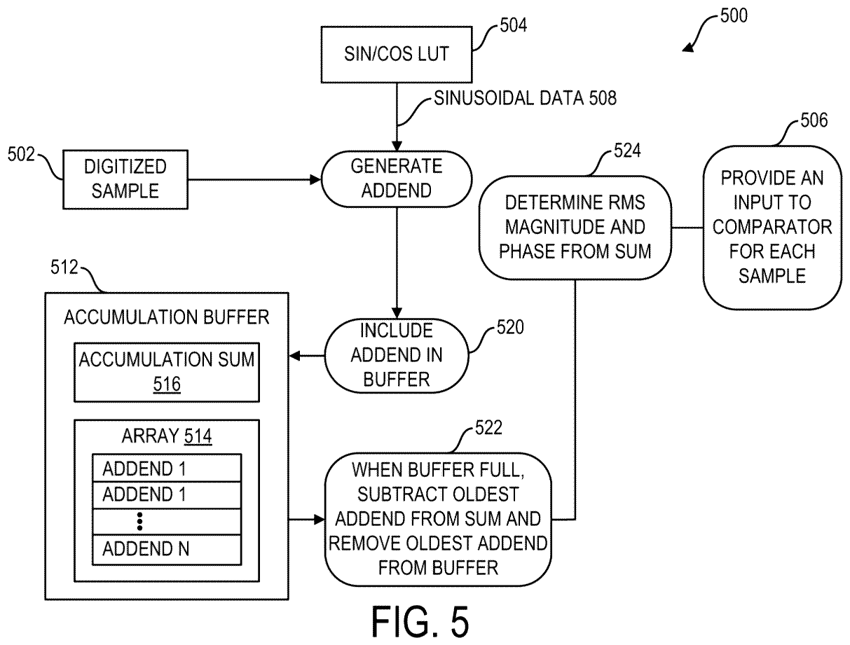

FIG. 5 illustrates a process 500 of the closed loop controller 300 configured according to process 400 such as to adjust an amplitude of the RF energy signal provided to the active terminal of an electrosurgical generator 102, in which the adjusted amplitude based on a setpoint 308 and an input (feedback input) O_{M} in a closed feedback loop of controller 300 in FIG. 3. In one embodiment, the process 500 is implemented with a field programmable gate array of the output measurement mechanism 304 and is repeated with each sample of the RF energy signal received over time. Process 500 has access to the sinusoidal LUTs 504 associated with the target frequency of the RF energy signal. Accordingly, the sinusoidal LUTs 504 applied during the process 500 are associated with the frequency bin k and number of bins N. Process 500 receives as an input of a digitize sample of the RF energy signal 502 and an output value of each of the sine LUT and cosine LUT 508 corresponding with the index value incremented after each sample 502. In one embodiment, process 500 generates a magnitude and phase result that provide to the comparator 308 as an input 506 for each sample after N samples have been received at the output measurement mechanism 304. The input 506 is based on a rolling sum of a plurality of addends. Each addend of the plurality of addends is based on a sample of the RF energy signal 502 associated with the frequency bin, which is used to determine the output vales of sinusoidal LUTs. The rolling sum of addends are determined over the number of frequency bins N associated with the target frequency of the RF energy signal.

Process 500 generates a new addend for the rolling sum from the received RF energy signal 502 and output value of the sinusoidal LUTs 508 for every sample at 510. The value of the sample (VSAMPLE) in embodiments can be calibrated or uncalibrated (raw value measurement). In one embodiment, the new addend is determined based on the value of the sample (V_{SAMPLE}), the output from the cosine LUT (cos) and output from the sine LUT (sin) for the corresponding index according to:

$$new addend = {V}_{SAMPLE} * ( cos + j * sin )$$

in which j is the square root of (−1).

The new addend is included in a rolling accumulation buffer 512 for each received sample 502 at 520. In the embodiment, the rolling accumulation buffer 512 includes an array 514 having a size of N addends, which are arranged from oldest sample to newest sample. The rolling accumulation buffer 512 also stores a rolling accumulation sum 516, which is equal to the sum of all of the addends in the array 514 added together. For example, if the addends received in order of samples in time are 0, 1, 1, 2, 3, 5, the rolling accumulation buffer 512 would arrange the addends as “0” being the oldest sample, “5” being the newest sample, and so on, in the array 514. The value stored in the rolling accumulation sum 516 is 12 (0+1+1+2+3+5=12). If a new addend is included to the rolling accumulation buffer 512 at 520 once the array 514 includes N addends, the oldest addend is popped from the array 514 and subtracted from the rolling accumulation sum 516 for each received sample 502 at 522. Accordingly, the rolling accumulation buffer 512 includes at most the latest N addends in array 514 arranged in order from oldest sample and newest sample, and the rolling sum 516 of the latest N addends as operative values in process 500. In some embodiments, the rolling accumulation buffer 512 includes multiple sub-buffers wherein each sub-buffer can store and sum components of the addends. For example, a rolling accumulation buffer can include a first sub buffer to store and sum real components of the addends and a second sub-buffer to store and sum (j) imaginary components of the addends. The sums in the first and second buffers can be combined to produce the real and (j) imaginary components of the rolling accumulation sum 516. In one embodiment, the rolling accumulation buffer 512, including the sub-buffers, are implemented as a data structure.

Once the rolling accumulation buffer has reached N addends in the array 514 and in the sum 516, the sum 516 is processed to determine signal RMS magnitude and phase information at 524 for each received sample 502. In some embodiments, just RMS magnitude is determined and provided to the comparator 308 without phase information. In one example, the RMS magnitude is determined according to the RMS of the rolling accumulation sum 516 Magnitude (sqrt(real²+imaginary²)) divided by N, and the phase is determined from the arctangent of the rolling accumulation sum 516. The magnitude function, Mag( ), calculates the length or magnitude of a vector, and sqrt( ) is the square root function. The RMS magnitude and phase information are provided to the comparator 308 in the feedback loop controller 300 as a feedback input 506.

In some embodiments, the following pseudo code describes an implementation of process 500 to generate an input to the comparator 308 with a digitized sample of the RF energy signal using the sinusoidal LUTs 504 and the rolling accumulation buffer 512 with addends 514 and rolling sum 516.

```
%Perform in controller for each sample received, sample by sample
while (data incoming)
  %extract sinusoidal data from tables
  sinLoopUpSpot = sinTable(sinusoidCounter);
  cosLoopUpSpot = cosTable(sinusoidCounter);
  %reset sinusoidCounter after each cycle
```

| | |
|---|---|
| if | (sinusoidCounter > size of tables) |
| | sinusoidCounter = 0 |

else

sinusoidCounter = sinusoidCounter + 1;

```
  end if
  % Output RMS magnitude and phase to comparator
```

| | |
|---|---|
| if | (rollingBuffer is FULL) |
| | out Signal_RMS(timepoint) = Mag(buffer_Sum) * 2/sqrt(2)/N; |
| | out Signal_Phase(timepoint) = arctan(buffer_Sum); |
| | %remove oldest entry from buffer and accumulation sum |
| | pop oldestBufferEntry from rollingBuffer |
| | buffer_Sum = buffer_Sum − (oldestBufferEntry) |

```
  end if
  %create new addend
  newBufferEntry = new_Sample * ( cosLoopUpSpot + j*sinLoopUpSpot );
  %include new addend into buffer and accumulation sum
  push newBufferEntry onto rollingBuffer
  buffer_Sum = buffer_Sum + newBufferEntry;
loop
```

The process 500 accesses the sinusoidal data, which is based on bin number k and number of bins N, generated at 406 and kept in LUTs 504 sin Table(sinusoidCounter) and cos Table(sinusoidCounter). Each new addend newBufferEntry is based on sampled output new_Sample associated with sinusoidal data sin LookUpSpot and cos LookUpSpot as determined by an index, in which the index repeats after each cycle. The number of indexes sinusoidCounter can differ from the number of frequency bins N, so the index resets separately from the number of addends in the array 514. The new addend is included into the rolling accumulation buffer at 520 as it is pushed into the array 514 at push newBufferEntry onto rollingBuffer and added to the rolling accumulation sum 516 at buffer_Sum=buffer_Sum+newBufferEntry. When the buffer is full, or there are N addends in the array 514, the oldest addend in the array 514 is removed from the array pop oldestBufferEntry from rollingBuffer and Subtracted from the rolling accumulation sum 516 buffer_Sum=buffer_Sum−(oldestBufferEntry) at 524. At each sample, an feedback input is generated to include RMS magnitude out Signal_RMS(timepoint)=Mag(buffer_Sum)*2/sqrt(2)/N and phase out Signal_Phase(timepoint)=arctan(buffer_Sum), which feedback input is provided to the comparator 308 and applied to the setpoint 306 of the feedback loop controller 300.

The feedback loop controller 300 with the output measurement mechanism 304 determined via process 500 is extremely accurate, fast, and include high resolution of a captured and processed RF energy signal. In one example, the feedback input is provided to the comparator at a rate of 1 MHz. In one measurement, a tissue crossing can be performed in 1 millisecond, which would include 1,000 samples of process 500 during a crossing. Process 500 develops magnitude and phase information that can be applied by other features of the generator 200, which eliminates the need for additional electronic circuitry such as filters to determine such information from the RF energy signal.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical generator, comprising:
a plurality of device terminals including an active terminal and a return terminal; and
a controller configured to:
adjust an amplitude of a radiofrequency (RF) energy signal provided to the active terminal, the adjusted amplitude based on a setpoint and an input in a closed feedback loop,
wherein the input is based on a rolling sum of a plurality of addends, each addend of the plurality of addends based on a sampled output of the RF energy signal associated with a frequency bin, the rolling sum of addends determined over a number of frequency bins associated with a target frequency.

2. The electrosurgical generator of claim 1, wherein the controller further includes a feedback control device to receive an error based on the setpoint and the input.

3. The electrosurgical generator of claim 2, wherein the feedback control device includes a proportional-integral-derivative (PID) mechanism.

4. The electrosurgical generator of claim 2, wherein the feedback control device is configured to adjust the amplitude of the RF energy signal.

5. The electrosurgical generator of claim 1, wherein the frequency bin and the number of frequency bins are applied to determine the target frequency.

6. The electrosurgical generator of claim 5, wherein the frequency bin and the number of frequency bins are applied to determine a sampling rate of the sampled output.

7. The electrosurgical generator of claim 1, wherein the frequency bin and the number of frequency bins are applied to determine sinusoidal data applied to the sampled output to determine each addend.

8. The electrosurgical generator of claim 7, wherein the sinusoidal data is received from a look up table.

9. The electrosurgical generator of claim 8, wherein the look up table includes a cycle of sinusoidal data.

10. The electrosurgical generator of claim 1, wherein the rolling sum of the plurality of addends is based on the plurality of addends determined over time.

11. The electrosurgical generator of claim 10, wherein the plurality of addends is equal to the number of frequency bins.

12. The electrosurgical generator of claim 1, wherein the plurality of addends and the rolling sum of the plurality of addends are received from a rolling accumulation buffer.

13. The electrosurgical generator of claim 1, wherein the input includes a magnitude and phase based on the rolling sum of the plurality of addends.

14. The electrosurgical generator of claim 13, wherein the magnitude is a root-mean-square of the rolling sum of the plurality of addends.

15. The electrosurgical generator of claim 14, wherein the phase is an arctangent of the rolling sum of the plurality of addends.

16. An electrosurgical system, comprising:
an electrosurgical device having an active electrode; and
an electrosurgical generator, comprising:
a plurality of device terminals including an active terminal and a return terminal, the active electrode configured to electrically couple to the active electrode; and
a controller configured to:
adjust an amplitude of a radiofrequency (RF) energy signal provided to the active terminal, the adjusted amplitude based on a setpoint and an input in a closed feedback loop,
wherein the input is based on a rolling sum of a plurality of addends, each addend of the plurality of addends based on a sampled output of the RF energy signal associated with a frequency bin, the rolling sum of addends determined over a number of frequency bins associated with a target frequency.

17. The electrosurgical system of claim 16, wherein the electrosurgical device includes a crossing device.

18. The electrosurgical system of claim 17, wherein the crossing device includes a transseptal guidewire.

19. A method for use with an electrosurgical generator having a plurality of device terminals including an active terminal, the method comprising:
adjusting an amplitude of a radiofrequency (RF) energy signal provided to the active terminal, the adjusted amplitude based on a setpoint and an input in a closed feedback loop; and
wherein the input is based on a rolling sum of a plurality of addends, each addend of the plurality of addends based on a sampled output of the RF energy signal associated with a frequency bin, the rolling sum of addends determined over a number of frequency bins associated with a target frequency.

20. The method of claim 19, and further comprising receiving sinusoidal data from a lookup table, the sinusoidal data applied to the sample output to determine each addend.
